(19) United States
(12) Patent Application Publication
(10) Pub. No.: US 2009/0125241 A1
(43) Pub. Date: May 14, 2009

(54) REAL TIME WATER ANALYSIS SYSTEM FOR METALS, CHEMICALS, RADIOLOGICAL AND BIOLOGICAL MATERIALS (CBRNME) WITHIN WATER

(75) Inventor: David L. Frank, Boca Raton, FL (US)

Correspondence Address: FLEIT GIBBONS GUTMAN BONGINI & BIANCO P.L., ONE BOCA COMMERCE CENTER, 551 NORTHWEST 77TH STREET, SUITE 111, BOCA RATON, FL, 33487, US

(73) Assignee: Innovative American Technology, Inc., Boca Raton, FL (US)

(21) Appl. No.: 11/931,211

(22) Filed: Oct. 31, 2007

Related U.S. Application Data
(63) Continuation-in-part of application No. 11/564,193, filed on Nov. 28, 2006.
Application No. 11/564,193 is a continuation-in-part of application No. 11/291,574, filed on Dec. 1, 2005.
Application No. 11/291,574 is a continuation-in-part of application No. 10/280,255, filed on Oct. 25, 2002, now Pat. No. 7,005,982.
(60) Provisional application No. 60/861,842, filed on Nov. 29, 2006.
(60) Provisional application No. 60/966,703, filed on Aug. 29, 2007.
(60) Provisional application No. 60/878,861, filed on Jan. 5, 2007.
(60) Provisional application No. 60/759,332, filed on Jan. 17, 2006.
(60) Provisional application No. 60/759,331, filed on Jan. 17, 2006.
(60) Provisional application No. 60/759,373, filed on Jan. 17, 2006.
(60) Provisional application No. 60/759,375, filed on Jan. 17, 2006.
(60) Provisional application No. 60/347,997, filed on Oct. 26, 2001.
(60) Provisional application No. 60/631,865, filed on Dec. 1, 2004.
(60) Provisional application No. 60/655,245, filed on Feb. 23, 2005.

(51) Int. Cl. G01N 33/48; G01N 30/02; H01J 49/26; G01K 17/00; G01N 31/00

(52) U.S. Cl. 702/19; 73/23.35; 250/282; 374/31; 702/22; 374/E17.001

(57) ABSTRACT

A method and water analysis system are provided to automatically, and without manual intervention, detect and identify contamination and/or hazardous material within one or more water samples from a potable and/or effluent water system. The method includes collecting a water sample from a potable and/or effluent water system; monitoring, in response to the collecting, sensors-detectors that are located in proximity to the collected water sample and receiving sensor-detector data from the sensors-detectors. The sensors-detectors include: laser induced breakdown spectrometry (LIBS) sensor technology, gas chromatography sensor technology, mass spectroscopy sensor technology, calorimetric spectroscopy sensor technology, and radiation detection technology. The method further includes spectrally analyzing, in response to the monitoring, the received sensor-detector data to detect, identify, and quantify, metals, chemicals, radiological materials, and biological materials, within the collected water sample.

### CROSS-REFERENCE TO RELATED APPLICATIONS

This application is based on, and claims priority from, prior co-pending U.S. Provisional Patent Application No. 60/861,842, filed on Nov. 29, 2006, by inventor David L. FRANK, and entitled “REMOTE SENSOR NETWORK FOR REAL-TIME ANALYSIS OF WATER SYSTEMS”; and further is based on, and claims priority from, prior co-pending U.S. Provisional Patent Application No. 60/966,703, filed on Aug. 29, 2007, by inventor David L. FRANK, and entitled “REAL-TIME WATER ANALYSIS SYSTEM FOR METAL, CHEMICALS AND BIOLOGICAL MATERIALS WITHIN WATER”; and further is based on, and claims priority from, prior co-pending U.S. Provisional Application No. 60/878,861, filed on Jan. 17, 2007, and entitled “Advanced Calorimetric Spectroscopy for Commercial Applications of Chemical and Biological Sensors”, and furthermore is a continuation-in-part of, and claims priority from, prior co-pending U.S. patent application Ser. No. 11/564,193, filed on Nov. 28, 2006, which is a continuation-in-part of, and claims priority from, prior co-pending U.S. patent application Ser. No. 11/291,574, filed on Dec. 1, 2005, which is based on, and claims priority from, prior co-pending U.S. Provisional Patent Application No. 60/759,332, filed on Jan. 17, 2006, by inventor David L. FRANK, and entitled “Sensor Interface Unit And Method For Automated Support Functions For CBRNE Sensors”; and further which is based on, and claims priority from, prior co-pending U.S. Provisional Patent Application No. 60/759,331, filed on Jan. 17, 2006, by inventor David L. FRANK, and entitled “Method For Determination Of Constituents Present From Radiation Spectra And, If Available, Neutron And Alpha Occurrences”; and further is based on, and claims priority from, prior co-pending U.S. Provisional Patent Application No. 60/759,373, filed on Jan. 17, 2006, by inventor David L. FRANK, and entitled “Distributed Sensor Network with Common Platform for CBRNE Devices; and further is based on, and claims priority from, prior co-pending U.S. Provisional Patent Application No. 60/759,375, filed on Jan. 17, 2006, by inventor David L. FRANK, and entitled Advanced Container Verification System; and wherein prior co-pending U.S. patent application Ser. No. 11/291,574, filed on Dec. 1, 2005, is a continuation-in-part of, claims priority from, prior co-pending U.S. patent application Ser. No. 10/280,255, filed on Oct. 25, 2002 and now U.S. Pat. No. 7,005,982 issued on Feb. 28, 2006, that was based on prior U.S. Provisional Patent Application No. 60/347,997, filed on Oct. 26, 2001, now expired, and which further is based on, and claims priority from, prior co-pending U.S. Provisional Patent Application No. 60/631,865, filed on Dec. 1, 2004, now expired, and which furthermore is based on, and claims priority from, prior co-pending U.S. Provisional Patent Application No. 60/655,245, filed on Feb. 23, 2005, now expired, and which furthermore is based on, and claims priority from, prior co-pending U.S. Provisional Patent Application No. 60/849,350, filed on Oct. 4, 2006, and which furthermore is based on, and claims priority from, prior co-pending U.S. patent application Ser. No. 11/363,594, filed on Feb. 27, 2006 and now U.S. Pat. No. 7,142,109 issued on Nov. 28, 2006; the entire collective disclosure of all the above-identified applications being hereby incorporated by reference.

### BACKGROUND OF THE INVENTION

1. Field of the Invention

The present invention generally relates to the field of water analysis systems, and more particularly relates to a water analysis system that operates automatically and without manual intervention for an extended period of test cycles to detect, identify, and quantify, metals, chemicals, radiological materials, and biological materials, within water samples.

2. Description of Related Art

Water systems are vulnerable to industrial pollutants, inadvertent and purposeful spills, pollutants from agriculture such as pesticides, and the very real possibility of the introduction of hazardous materials through terrorist activity.

Water systems are routinely sampled but very infrequently continuously monitored.

Detection of hazardous compounds or materials in water systems typically occurs after the contaminants have been allowed to flow through the systems for days, weeks, or even months.

Current methods for water analysis use laboratory devices that require calibration and water sample preparation methods that required manual intervention. In most cases, manual intervention is performed by a highly skilled worker to ensure proper operation. Such devices and methods are not practical for field deployment in a real-time and continuous operation.

Detecting the presence of organic compounds, metals, radiological and biological materials in potable and effluent water systems is a matter of either periodic manual processes, the discovery of a catastrophic event such as the death of fish used to monitor potable systems, the destruction of the micro organisms used to clean effluent systems, or the observation of harmful environmental impacts.

Once detected it is often difficult to determine where or when the contamination occurred.

Therefore a need exists to overcome the problems with the prior art as discussed above.

### SUMMARY OF THE INVENTION

According to one embodiment of the present invention, a method is provided for automatic detection and identification of contamination and/or hazardous material within one or more water samples from a potable and/or effluent water system. The method comprises:
collecting, automatically and without manual intervention, a water sample from a potable and/or effluent water system;
monitoring, automatically and without manual intervention, in response to the collecting, a plurality of sensors-detectors that are located in proximity to the collected water sample and receiving sensor-detector data from the plurality of sensors-detectors, the plurality of sensors-detectors including:
   laser induced breakdown spectrometry (LIBS) sensor technology;
   gas chromatography sensor technology,
   mass spectroscopy sensor technology,
   calorimetric spectroscopy sensor technology, and
   radiation detection technology; and
spectrally analyzing, automatically and without manual intervention, in response to the monitoring, the received sensor-detector data to detect, identify, and quantify, metals, chemicals, radiological materials, and biological materials, within the collected water sample.

In accordance with a second embodiment of the present invention, a water analysis system comprises:
a water flow controller for automatically and without manual intervention controlling the collection of a water sample from a potable and/or effluent water system;
a plurality of sensors-detectors for locating in proximity to the collected water sample and receiving sensor-detector data from the plurality of sensors-detectors, the plurality of sensors-detectors including:
   laser induced breakdown spectrometry (LIBS) sensor technology;
   gas chromatography sensor technology,
   mass spectroscopy sensor technology,
   calorimetric spectroscopy sensor technology, and
   radiation detection technology; and
an information processing system, communicatively coupled with the water flow controller and the plurality of sensors-detectors, the information processing system being adapted to:
   collect, automatically and without manual intervention, a water sample from a potable and/or effluent water system;
   monitor, automatically and without manual intervention, in response to the collecting, the plurality of sensors-detectors that are located in proximity to the collected water sample and receive sensor-detector data from the plurality of sensors-detectors; and
   spectrally analyze, automatically and without manual intervention, in response to the monitoring, the received sensor-detector data to detect, identify, and quantify, metals, chemicals, radiological materials, and biological materials, within the collected water sample.

### BRIEF DESCRIPTION OF THE DRAWINGS

The accompanying figures where like reference numerals refer to identical or functionally similar elements throughout the separate views, and which together with the detailed description below are incorporated in and form part of the specification, serve to further illustrate various embodiments and to explain various principles and advantages all in accordance with the present invention.

FIG. 1 is a block diagram illustrating an example of a water analysis system according to one embodiment of the present invention.

FIG. 2 is an operational flow diagram illustrating an operational sequence of the water analysis system of FIG. 1.

FIG. 3 is a block diagram illustrating a more detailed view of a laser induced breakdown spectroscopy (LIBS) sensor system used in the example of FIG. 1, according to one embodiment of the present invention.

FIG. 4 is a block diagram illustrating an example of several water analysis sensors for use in the water analysis system of FIG. 1.

### DETAILED DESCRIPTION OF THE INVENTION

As required, detailed embodiments of the present invention are disclosed herein; however, it is to be understood that the disclosed embodiments are merely examples of the invention, which can be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure. Further, the terms and phrases used herein are not intended to be limiting; but rather, to provide an understandable description of the invention.

The terms “a” or “an”, as used herein, are defined as one or more than one. The term plurality, as used herein, is defined as two or more than two. The term another, as used herein, is defined as at least a second or more. The terms including and/or having, as used herein, are defined as comprising (i.e., open language). The term coupled, as used herein, is defined as connected, although not necessarily directly, and not necessarily mechanically.

The present invention, according to various embodiments, provides a real time and continuous water analysis system to detect, identify, and quantify, hazardous materials, such as metals, chemicals, radiological material, and biological materials, within a water sample, and without manual intervention for an extended period of test cycles.

This system uses an open architecture for a distributed network connecting a wide variety of CBRNME sensors, such as disclosed in U.S. Pat. No. 7,005,982, by inventor David L. Frank, and entitled “DISTRIBUTED SENSOR NETWORK WITH COMMON PLATFORM FOR CBRNME DEVICES”, the entire disclosure thereof being hereby incorporated by reference.

The use of multiple sensors and sensor types for analysis of water systems are described in Provisional Patent Application No. 60/861,842, entitled “REMOTE SENSOR NETWORK FOR REAL-TIME ANALYSIS OF WATER SYSTEMS”, and in Provisional Patent Application No. 60/966,703, entitled “REAL-TIME WATER ANALYSIS SYSTEM FOR METAL, CHEMICALS AND BIOLOGICAL MATERIALS WITHIN WATER”; the entire collective disclosure of the above-identified applications being hereby incorporated by reference.

A water analysis system, according to one example, uses a combination of sensors tailored for the type of material to be detected, and transmits data back to an operations center for analysis, and in response to the analysis for generating alarms and implementing response protocols, as will be discussed in more detail below.

Alarm criterion and response protocols can be uniquely defined for each water analysis system and may further be uniquely defined for each location within the system. The sensors used in such a system may be monitored locally, remotely, or both.

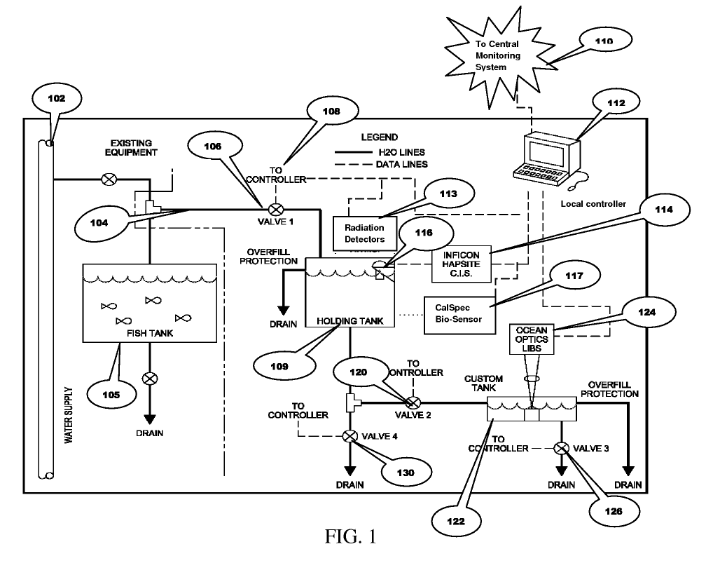

The example of the system depicted in FIG. 1, shows one station in a multiple station system. In this example, each station is capable of locally analyzing the data and locally executing business rules that have been defined for that location. In various examples, the local station transmits its findings and data to a central monitoring system, and to any other location as may be desired by the user.

The system provides a user interface through which the user may specify; the types of tests to be conducted, the number of test cycles per day, the set of business rules, by location, that are to be executed in the event of an alarm such as; notify lab personnel via pager, notify plant management, etc., the criterion which determines an alarm, per substance, such as; x parts per billion (ppb) of benzene, or y ppb of cesium, etc. Once the rules are established they are continually used until changed by authorized users.

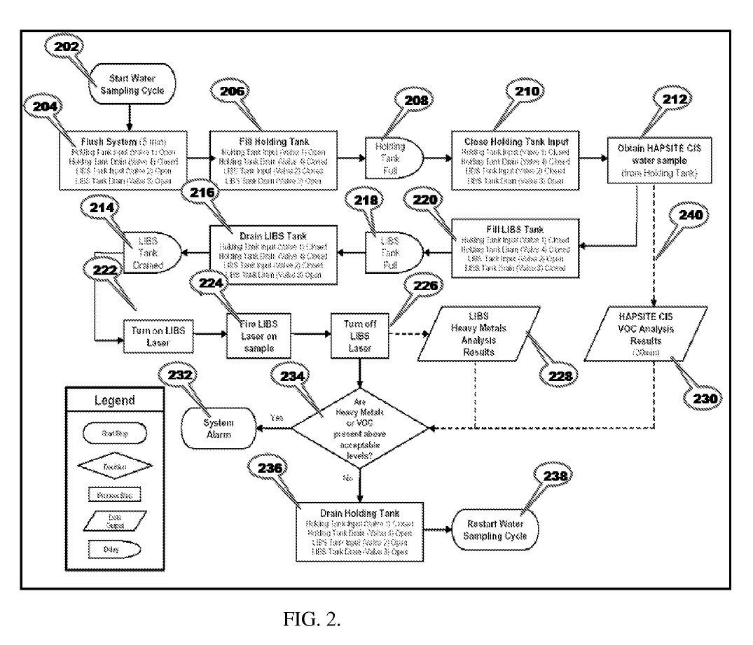

This water analysis system includes a unique sample delivery system to enable continuous test cycles. One example of this is depicted in FIG. 1, which shows the water sample capture and analysis mechanism for a potable water system, and FIG. 2, which shows a process used to capture and test the water sample.

In the example depicted in FIG. 1, the particular water analysis system uses various sensor/detector systems, including: 1) a commercial-off-the-shelf (COTS) laser induced breakdown spectroscopy (LIBS) sensor, such as from Ocean Optics Corporation, for the detection of metals, 2) a COTS Gas Chromatography and Mass Spectrometry (Hapsite™) system, such as from Inficon Corporation, for the detection of volatile organic compounds, 3) an Advanced Calorimetric Spectroscopy (CalSpec) sensor system available from Innovative American Technologies Corporation, and as described in U.S. Provisional Patent Application No. 60/878,861, filed on Jan. 17, 2007, and entitled “Advanced Calorimetric Spectroscopy for Commercial Applications of Chemical and Biological Sensors”, (the entire teachings of which being hereby incorporated by reference), for detection of semi-volatile organic compounds and for the detection of biological compounds, and 4) one or more COTS radiation detectors for detection of radiological materials; all sensor/detector systems being communicatively coupled with an information processing system via a common sensor interface platform.

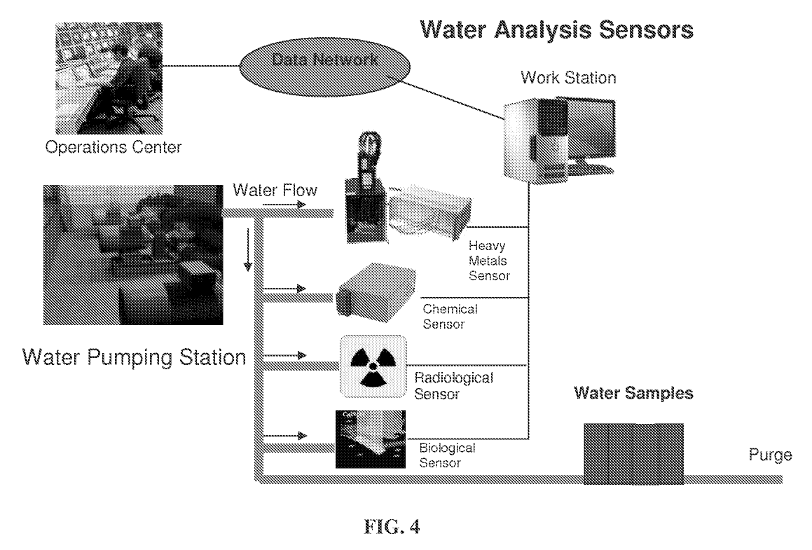

An overview of such an information processing system communicatively coupled to various sensor/detector systems to implement a water analysis system is illustrated in FIG. 4. The information processing system, in this example, comprises a workstation with one or more processors. The information processing system is also communicatively coupled, via a data network, to an operations center that may be located remotely to the water analysis system. Water from a pumping station is delivered, under control of the information processing system, to the various sensor/detector systems, including a heavy metals sensor, a chemical sensor, a radiological sensor, and a biological sensor. The water can be delivered in controlled water samples that after testing are purged out of the water analysis system. The water analysis system can operate automatically and without human intervention to test and analyze water samples over an extended period of test cycles. This repeating set of test cycles can last hours, days, weeks, or months, depending on the particular requirements of a water analysis system application.

This information processing system uses spectral data analysis software, such as described in U.S. Pat. No. 6,847,731, entitled “Margin Setting”, and as described in U.S. Provisional Patent Application No. 60/759,337, entitled “Advanced Pattern Recognition System”, (the entire collective teachings of the above-identified patent and provisional patent application being hereby incorporated by reference), for chemical, biological, and radiological spectral signature identification. Upon spectral signature identification, such as when a match is detected between a known signature and at least a portion of a spectral image captured from a test sample, the water analysis system can automatically trigger appropriate action with the information processing system, such as sending an alarm to user(s), e.g., authorized personnel, and executing local business rules, and further communicating data that relates to the detected situation with a central monitoring system (e.g., an operations center). For example, the data can include sensor-detector system status and associated sensor-detector data, real-time monitored environmental conditions about the particular monitored location of the water analysis system, and data associated with pre-defined business rules associated with a specific spectral signature identification.

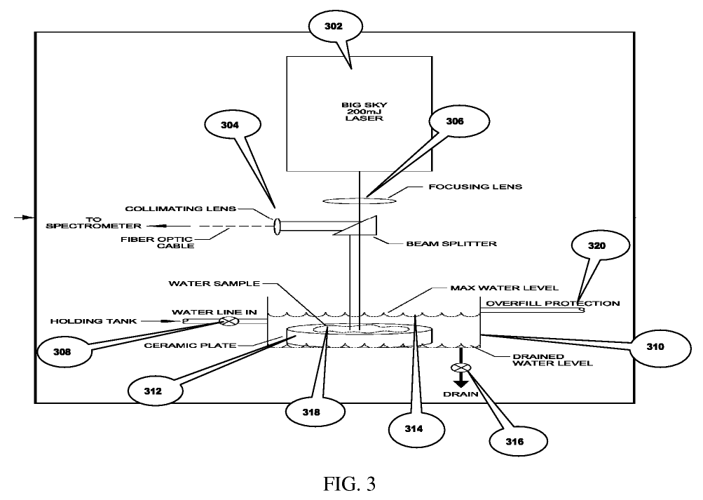

A more detailed description of a sample delivery system for a LIBS analysis system, as described in U.S. Provisional Patent Application No. 60/966,703, entitled “Real-Time Water Analysis System for Metal, Chemicals and Biological Materials Within Water” is depicted in FIG. 3.

Described now, with reference to FIGS. 1, 2, and 3, is one example of a water analysis system that uses multiple sensors and multiple sensor types to provide for real-time and continuous automatic monitoring of potable and effluent water systems. The water analysis system can operate unattended, with no manual intervention for weeks at a time. The system is automatically, and without manual intervention, self-calibrating and can perform multiple test cycles per day.

This particular example, described with reference to FIGS. 1 through 3, includes the placement of a Hapsite™ system for detection of volatile organic compounds and a LIBS system for detection of metals in a potable water distribution system.

FIG. 1 of this example depicts the unique sample delivery system to accommodate the requirements of the two sensor types being used in this application. FIG. 2 shows a process flow of this particular application. FIG. 3 shows a more detailed view of the LIBS environment.

The entire test cycle is controlled by the local controller/processor 112, which in this example is a COTS information processing server system running both client and server software to control all local processing such as: managing the local network (shown as dotted lines) 108, opening and closing of valves, accepting data from the various sensors, analysis of the test samples, execution of local business rules, monitoring the health of the various sensors, and communicating with a central monitoring system 110 over a data network, such as using standard TCP/IP protocol. The data network can include one or more local area networks and one or more wide area networks, such as the Internet and the world-wide-net. It can also include any combination of wired and wireless communications.

With respect to TCP/IP communications, one implementation of a water analysis system includes a plurality of sensors-detectors that each is individually identified by a TCP/IP address. A sensor interface unit (SIU) is communicatively coupled with the plurality of sensors-detectors and also communicatively coupled with a monitoring information processing system. The SIU maintains TCP/IP address information for the plurality of sensors-detectors, and, in one embodiment, associates each of the plurality of sensors-detectors individually with a TCP/IP address. This SIU and sensors-detectors interface arrangement is described in more detail in U.S. patent application Ser. No. 11/564,193, filed on Nov. 28, 2006, which is hereby incorporated by reference. When sensor-detector data associated with a particular sensor-detector is received from the sensors-detectors, it then is sent to a monitoring information processing system via TCP/IP communications over a data network. Such sensor-detector data can be sent, according to one embodiment, in response to receiving a request for such sensor-detector data associated with a TCP/IP address. For example, an information processing system can request particular sensor-detector data from one or more sensors-detectors. In response, this sensor-detector data, when available, is sent to the information processing system. To identify the particular sensor-detector data, the request is associated with one or more TCP/IP addresses which identify the individual sensors-detectors.

At the start of the Test Cycle 202, water is drawn in line 104 from the output line of the water distribution system 102 by opening valve 1 106. A water flow controller, according to one embodiment of the present invention, can control the one or more flow valves and/or pumping stations in particular applications. Such a flow controller can also be part of an information processing system. (The use of fish in an isolated tank 105 is common in potable water systems. It is illustrated here to show the relative placement of a test sample acquisition system.)

The first step in the process 204 is to flush the entire system. The flush time is user definable but normally is not less than five (5) minutes. To flush the system, valves 1 106, 2 120, and 3 126, are all opened, and valve 4 130 is closed; ensuring that the entire sample area is flushed including the stagnant LIBS sample 318. At the end of the flush interval, at step 2 of the process 206, the system fills the “Holding Tank” 109 by closing valve 2 120.

When the holding tank 109 is determined to be full 208, valve 1 106 is closed 210, isolating a test water sample. With the holding tank 109 full with an isolated test sample, at step 210, the Hapsite™ system 114 can begin its testing cycle. The Hapsite™ system 114 includes a combination gas chromatograph and mass spectrometer, that can detect and identify volatile organic compounds. The Hapsite™ system testing protocols can be tailored to user specifications, including what compounds to look for and in what quantity. The Hapsite™ situ-probe 116 is permanently mounted in the holding tank 109. The situ-probe 116 uses a gas extraction process to deliver a gas sample to the Hapsite™ system 114. Once the Hapsite™ system starts its test cycle 212, the “custom tank” 122, 310, can be filled, at step 220, using the water from the isolated sample.

To fill the “custom Tank” 122, 310, and to prepare the water sample 220 for the LIBS system 124, valve 2 120 is opened and valve 3 126, 316, is closed. The water in the custom tank 122, 310, is filled to the maximum water level 314, at step 218. This can be accomplished by a sensor to detect the water level (not shown) or by a measured flow over time, which is the method used as an underlying assumption in this example. When the LIBS custom tank 122, 310, is determined to be full, at step 218, the tank is drained 216 by opening valve 3 126, 316. When the LIBS custom tank 122, 310, is determined drained, at step 214, this leaves a small sample of water on the ceramic plate 312 that is permanently fixed in the tank 122, 310, and positioned under the laser 302. This small sample is the stagnant sample 318 used by the LIBS system 124. The laser 302 is turned on 222 and fired 224 at the water sample 318 through a focusing lens 306. The resulting spectral image is captured by the collimating lens 304 for analysis by the LIBS spectrometer, at steps 226, 228.

At the completion of the test cycle by the Hapsite™ 230 and LIBS 228 they communicate their findings via the local network (dotted lines 240, 108) to the local controller/processor 112 where the results are processed against the local business rules 234. For example, these local business rules can be stored as records in a local data base that is communicatively coupled with the local controller/processor 112. If an alarm condition is detected 232, the isolated test sample is retained for further testing and analysis. If there is no alarm condition 236 the local controller/processor 112 sends a signal to open valve 4 130 which drains the holding tank 109. In either case the local server 112 communicates the result of the test to the central alert notification server 110 with a time-stamped record of the test, the results and actions taken. This record is also stored locally in the local data base for future reference as may be necessary.

At this point, the system is ready to begin a new test cycle, at step 238. The test cycle intervals can be defined by the user.

By automatically, and without manual intervention, over an extended period of test cycles, testing water samples, the water analysis system according to the present invention provides significant advantages not previously available by any known water analysis systems. It can provide near real-time monitoring and response to monitored conditions, with prompt generation of alarms to personnel and with automatic implementation of pre-defined business rules that can be customized for particular applications.

Although specific embodiments of the invention have been disclosed, those having ordinary skill in the art will understand that changes can be made to the specific embodiments without departing from the spirit and scope of the invention. The scope of the invention is not to be restricted, therefore, to the specific embodiments, and it is intended that the appended claims cover any and all such applications, modifications, and embodiments within the scope of the present invention.

What is claimed is:

1. A method for automatic detection and identification of contamination and/or hazardous material within one or more water samples from a potable and/or effluent water system, the method comprising:
collecting, automatically and without manual intervention, a water sample from a potable and/or effluent water system;
monitoring, automatically and without manual intervention, in response to the collecting, a plurality of sensors-detectors that are located in proximity to the collected water sample and receiving sensor-detector data from the plurality of sensors-detectors, the plurality of sensors-detectors including:
laser induced breakdown spectrometry (LIBS) sensor technology;
gas chromatography sensor technology,
mass spectroscopy sensor technology,
calorimetric spectroscopy sensor technology, and
radiation detection technology; and
spectrally analyzing, automatically and without manual intervention, in response to the monitoring, the received sensor-detector data to detect, identify, and quantify, metals, chemicals, radiological materials, and biological materials, within the collected water sample.

2. The method of claim 1, further comprising:
delivering, automatically and without manual intervention, a water sample to a water container that includes a raised platform therein, the water sample being delivered into the water container such that it attains a water level above a top surface of the raised platform and then the water level is lowered in the water container to provide a water sample residue on the top surface of the raised platform.

3. The method of claim 2, further comprising:
analyzing, using a LIBS analysis, automatically and without manual intervention, in response to the delivering, the water sample residue on the top surface of the raised platform.

4. The method of claim 2, further comprising:
cleaning, automatically and without manual intervention, the top surface of the raised platform by raising the water level in the water container and flushing with water the top surface of the raised platform.

5. The method of claim 1, wherein the collecting, monitoring, and spectrally analyzing, are performed automatically and without manual intervention, all under control of a controller/processor.

6. The method of claim 1, further comprising:
sending the received sensor-detector data to a monitoring information processing system via digital data packets using TCP/IP communications over a data network.

7. The method of claim 6, wherein each of the plurality of sensors-detectors is identified by a TCP/IP address, and wherein received sensor-detector data associated with a particular sensor-detector in the plurality of sensors-detectors is sent to the monitoring information processing system via TCP/IP communications over a data network, in response to receiving a request for such received sensor-detector data associated with a TCP/IP address.

8. The method of claim 1, further comprising:
generating an alarm, automatically and without manual intervention, in response to determining that the spectrally analyzed received sensor-detector data indicates contamination and/or hazardous material being detected in the water sample.

9. The method of claim 1, further comprising:
executing a set of business rules, automatically and without manual intervention, in response to determining that the spectrally analyzed received sensor-detector data indicates contamination and/or hazardous material being detected in the water sample.

10. A water analysis system comprising:
a water flow controller for automatically and without manual intervention controlling the collection of a water sample from a potable and/or effluent water system;
a plurality of sensors-detectors for locating in proximity to the collected water sample and receiving sensor-detector data from the plurality of sensors-detectors, the plurality of sensors-detectors including:
laser induced breakdown spectrometry (LIBS) sensor technology;
gas chromatography sensor technology,
mass spectroscopy sensor technology,
calorimetric spectroscopy sensor technology, and
radiation detection technology; and
an information processing system, communicatively coupled with the water flow controller and the plurality of sensors-detectors, the information processing system being adapted to:
collect, automatically and without manual intervention, a water sample from a potable and/or effluent water system;
monitor, automatically and without manual intervention, in response to the collecting, the plurality of sensors-detectors that are located in proximity to the collected water sample and receive sensor-detector data from the plurality of sensors-detectors; and
spectrally analyze, automatically and without manual intervention, in response to the monitoring, the received sensor-detector data to detect, identify, and quantify, metals, chemicals, radiological materials, and biological materials, within the collected water sample.

11. The water analysis system of claim 10, further comprising:
a communications device, communicatively coupled with the information processing system, to communicate sensor-detector data to a communications network, and wherein the information processing system is further adapted to:
send the received sensor-detector data to a monitoring information processing system via digital data packets using TCP/IP communications over a data network.

12. The water analysis system of claim 11, wherein the communications device comprises a sensor interface unit (SIU), and wherein each of the plurality of sensors-detectors is identified by a TCP/IP address maintained by the SIU, and further wherein received sensor-detector data associated with a particular sensor-detector in the plurality of sensors-detectors is sent to the monitoring information processing system via TCP/IP communications over a data network, in response to receiving a request for such received sensor-detector data associated with a TCP/IP address.

13. The water analysis system of claim 10, further comprising:
a water container that includes a raised platform therein; and
wherein the information processing system is further adapted to:
deliver, automatically and without manual intervention, the water sample to the water container, the water sample being delivered into the water container such that it attains a water level above a top surface of the raised platform and then the water level is lowered in the water container to provide a water sample residue on the top surface of the raised platform.

14. The water analysis system of claim 13, wherein the information processing system is further adapted to:
analyze, using a LIBS analysis, automatically and without manual intervention, in response to the delivering, the water sample residue on the top surface of the raised platform.

15. The water analysis system of claim 13, wherein the information processing system is further adapted to:
clean, automatically and without manual intervention, the top surface of the raised platform by raising the water level in the water container and flushing with water the top surface of the raised platform.

16. The water analysis system of claim 13, wherein the information processing system is further adapted to:
generate an alarm, automatically and without manual intervention, in response to determining that the spectrally analyzed received sensor-detector data indicates contamination and/or hazardous material being detected in the water sample.

17. The water analysis system of claim 13, wherein the information processing system is further adapted to:
execute a set of business rules, automatically and without manual intervention, in response to determining that the spectrally analyzed received sensor-detector data indicates contamination and/or hazardous material being detected in the water sample.
